(19) United States
(12) Patent Application Publication
(10) Pub. No.: US 2014/0094632 A1
(43) Pub. Date: Apr. 3, 2014

(54) METHODS AND APPARATUSES FOR RECOVERING NORMAL HEXANE FROM REFORMATE STREAMS

(71) Applicant: UOP LLC, Des Plaines, IL (US)

(72) Inventor: David James Shecterle, Arlington Heights, IL (US)

(73) Assignee: UOP LLC, Des Plaines, IL (US)

(21) Appl. No.: 13/631,442

(22) Filed: Sep. 28, 2012

(51) Int. Cl. C07C 7/04; C07C 2/00; C07C 7/144; B01J 8/00; C07C 7/13

(52) U.S. Cl. 585/400; 422/187; 585/700; 585/802

(57) ABSTRACT

Methods and apparatuses for recovering normal hexane from a reformate stream are provided. In one example, a method for recovering normal hexane from a reformate stream includes extracting aromatics from the reformate stream to form an aromatic extract stream and a raffinate stream. In the method, the normal hexane is separated from the raffinate stream to form a normal hexane product stream.

### TECHNICAL FIELD

The technical field relates generally to methods and apparatuses for processing hydrocarbons, and more particularly relates to methods and apparatuses for forming a normal hexane product from reformate streams.

### BACKGROUND

High octane gasoline is required for modern gasoline engines. Formerly, the octane number of hydrocarbons was improved by supplementing the hydrocarbons with lead-containing additives. When lead was phased out of gasoline for environmental reasons, it became necessary to rearrange the structure of the hydrocarbons used in gasoline blending to achieve higher octane ratings. Catalytic reforming is a widely used process for this refining of hydrocarbons to increase the yield of higher octane gasoline. In this process, paraffins and naphthenes are passed through a processing unit where their structure is rearranged to form higher octane aromatics while minimizing yield loss to cracking. Essentially catalytic reforming converts low octane paraffins to naphthenes. Naphthenes then are converted to higher octane aromatics.

The reforming process results in a reformate stream containing some aromatics and unconverted paraffins. In certain processing, the aromatics are extracted from the reformate stream to make higher value products. The raffinate stream formed by the aromatic extraction process will contain mostly paraffins, including C₆ paraffins. While normal hexane is difficult to separate from other C₆ paraffin isomers, it is a valuable solvent used in many industries, from food processing to bio-energy fields.

Accordingly, it is desirable to provide methods and apparatuses for recovering normal hexane from reformate streams. Furthermore, other desirable features and characteristics of the present disclosure will become apparent from the subsequent detailed description and the appended claims, taken in conjunction with the accompanying drawings and this background.

### SUMMARY

Methods and apparatuses for recovering normal hexane from a reformate stream are provided herein. In accordance with an exemplary embodiment, a method for recovering normal hexane from a reformate stream includes extracting aromatics from the reformate stream to form an aromatic extract stream and a raffinate stream. In the method, the normal hexane is separated from the raffinate stream to form a normal hexane product stream.

In accordance with another exemplary embodiment, a method forming a heavy aromatic rich product and a normal hexane product is provided. The method forming a heavy aromatic rich product and a normal hexane product includes reforming a hydrocarbon stream to form a reformate product from which a heavy aromatic rich product and a light reformate stream are formed. Non-aromatics are separated from the light reformate stream. The method isolates normal hexane from the non-aromatics to form a normal hexane product stream.

In accordance with another exemplary embodiment, an apparatus for recovering normal hexane from a reformate stream is provided. The apparatus includes a reforming unit configured to form the reformate stream. Further, the apparatus includes an aromatic extraction unit configured to extract aromatics from the reformate stream to form an aromatic extract stream and a raffinate stream. Also, the apparatus includes a separation unit configured to isolate normal hexane in a normal hexane product stream.

### BRIEF DESCRIPTION OF THE DRAWINGS

The method and apparatus for recovering normal hexane from a reformate stream will hereinafter be described in conjunction with the following drawing figures, wherein like numerals denote like elements, and wherein:

FIG. 1 schematically illustrates an apparatus and a method for forming a heavy aromatic rich product and a normal hexane product in accordance with an exemplary embodiment;

FIG. 2 schematically illustrates the apparatus and process of the hydrocarbon reforming zone of FIG. 1 in accordance with an exemplary embodiment;

FIG. 3 schematically illustrates the apparatus and process of the separation zone of FIG. 1 in accordance with an exemplary embodiment;

FIG. 4 schematically illustrates the apparatus and process of the aromatic extraction zone of FIG. 1 in accordance with an exemplary embodiment;

FIG. 5 schematically illustrates the apparatus and process of a normal hexane isolation zone of FIG. 1 in accordance with an exemplary embodiment;

FIG. 6 schematically illustrates the apparatus and process of an alternate normal hexane isolation zone of FIG. 1 in accordance with an exemplary embodiment;

FIG. 7 schematically illustrates the apparatus and process of an alternate normal hexane isolation zone of FIG. 1 in accordance with an exemplary embodiment;

FIG. 8 schematically illustrates the apparatus and process of an alternate normal hexane isolation zone of FIG. 1 in accordance with an exemplary embodiment; and

FIG. 9 schematically illustrates the apparatus and process of an alternate normal hexane isolation zone of FIG. 1 in accordance with an exemplary embodiment.

### DETAILED DESCRIPTION

The following Detailed Description is merely exemplary in nature and is not intended to limit the claimed apparatus or methods for recovering normal hexane from reformate. Furthermore, there is no intention to be bound by any theory presented in the preceding background or the following detailed description.

Methods and apparatuses for recovering normal hexane from a reformate stream are provided. As a result of the methods and apparatuses described herein, a reformate product, such as a high octane hydrocarbon stream, is formed by a reforming process. The reformate from the reforming process is processed to recover a normal hexane product. The normal hexane product can have substantially any desired concentration. However, to maximize yield of a valuable normal hexane product, the normal hexane product typically has a normal hexane concentration of more than about 40 wt %, for example more than about 50 wt % normal hexane, such as about 55 wt % normal hexane.

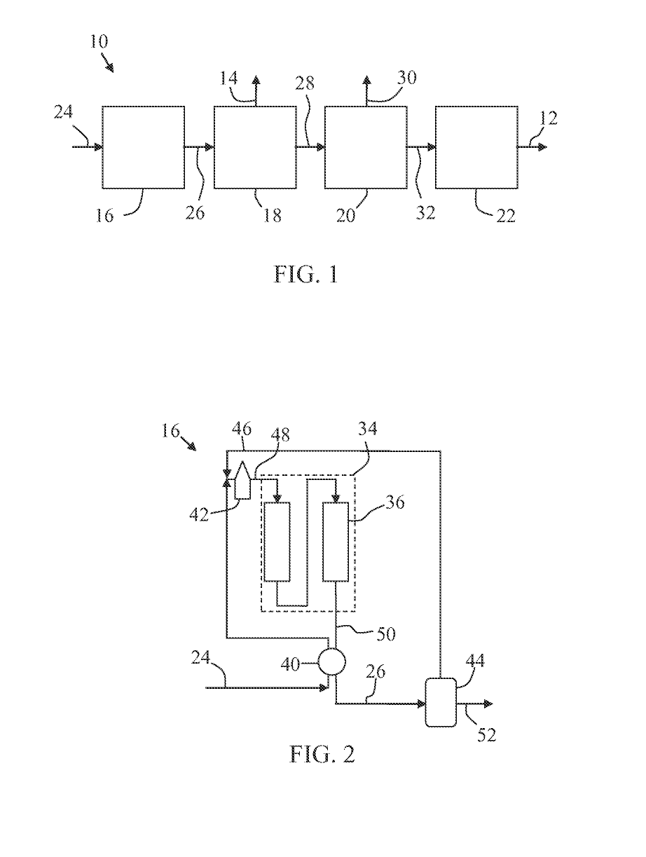

Referring to FIG. 1, an exemplary apparatus 10 for forming a normal hexane product stream 12 and a heavy aromatic rich product stream 14 is illustrated. As shown, the apparatus includes a reforming zone 16, a separation zone 18, an aromatic extraction zone 20 and a normal hexane isolation zone 22. During processing, a liquid hydrocarbon feedstock stream 24 is delivered to the reforming zone 16 where it is processed to increase its octane number. The resulting reformer effluent stream 26 is then delivered to the separation zone 18 where the heavy aromatic rich product stream 14 is separated from the remaining reformate stream 28. The reformate stream 28 is then fed to the aromatic extraction zone 20 where aromatics such as benzene, and toluene are removed to form an aromatic stream 30. The aromatic extraction zone 20 also forms a raffinate stream 32 which is delivered to the normal hexane isolation zone 22. There, the raffinate stream 32 is processed to separate the normal hexane product stream 12 with a desired normal hexane concentration, such as at least about 40 wt % normal hexane, for example at least about 50 wt % normal hexane or about 55 wt % normal hexane.

FIG. 2 provides a more detailed view of the reforming zone 16. As shown, the reforming zone 16 includes a reforming apparatus 34 that receives the liquid hydrocarbon feedstock stream 24. An exemplary liquid hydrocarbon feedstock stream 24 is rich in a C6-C10 hydrocarbon, such as hexane-decane, and an exemplary reforming apparatus 34 is a C6-C10 reforming apparatus. The reforming apparatus 34 may include one or more reforming units or reforming reactors 36, arranged in series or otherwise, that contain one or more reforming catalysts. Typically the reforming will be done in stages and the reforming apparatus 34 will include three or four reforming reactors 36. An exemplary reforming zone 16 can include a heat exchanger 40, a fired heater 42 immediately upstream of each reforming reactor 36, and a separator 44 to form a recycle gas 46.

As shown, the liquid hydrocarbon feedstock stream 24 enters the reforming zone 16 and passes through heat exchanger 40 where it is partially heated to reforming temperatures. The partially heated feedstock is then combined with recycle gas 46, comprising hydrogen and light (C₁-C₂) hydrocarbon gases, and the combined mixture is fed to fired heater 42 where it is further heated to reforming temperatures. The recycle gas 46 will typically be mixed with the liquid feedstock in proportions in the range of about three to about ten moles gas per mole of feedstock. The temperature of the feed to fired heater 42 will usually be about 260° C. and to about 425° C. (about 500° F. to about 800° F.); whereas the temperature of the heated effluent will usually range from about 450° C. to about 565° C. (from about 850° F. to about 1050° F.). The heated effluent 48 leaves the fired heater 42 and is introduced to reforming apparatus 34.

The reforming reactors 36 will generally include an active amount of a catalyst, such as a platinum group component, supported on a refractory porous carrier or base, such as high purity alumina. The catalyst will also preferably include a promoter that enhances the activity, fouling rate, stability and/or selectivity of the catalyst. The promoting agents normally employed are metals such as rhenium, germanium, and technetium. Reforming catalysts that contain such promoter metals are commonly called “bimetallic catalysts.” The platinum group component will usually comprise from 0.01% to 2%, more usually 0.1% to 1%, by weight calculated as metal and based on the finished catalyst. The promoter will usually be present in like proportions also calculated as metal and based on the finished catalyst. The finished catalyst also contains chloride from 0.1% to 2% by weight based on the finished catalyst.

The reforming reactors 36 will typically be operated at temperatures approximating the furnace effluent temperatures stated above and pressures in the range of about 2.74 to about 35.5 bar absolute (about 25 to about 500 psig), preferably about 4.46 to about 21.7 bar absolute (about 50 to about 300 psig), when a bimetallic catalyst is used. The temperature and pressure are correlated with liquid hourly space velocity (LHSV), e.g., volumes of liquid feed per hour processed per volume of catalyst, to provide the desired type of reforming. Generally the LHSV will be between about 0.1 and about 10 and more usually between 1 and 5.

The reformer effluent stream 26 is withdrawn from the reforming zone through line 50 and portions of the heat content thereof are exchanged to the liquid hydrocarbon feedstock stream 24 in heat exchanger 40. The reformer effluent stream 26 is typically cooled to about 90° C. to about 200° C. (about 200° F. to about 400° F.) by such exchange. After the heat exchange, the cooled reformer effluent stream 26 may be further cooled in a products condenser and then passed to a separator 44 where hydrogen-rich vapors are removed overhead for use as recycle gas 46. Hydrogen is provided to prevent or inhibit the formation of carbon by decomposition of the hydrocarbons during reforming. The reformer effluent stream 26 then exits the reforming zone 16 via line 52. Although not shown, it should be understood that fluid transfer devices, such as pumps and compressors, can be used to transport, respectively, the hydrocarbon liquid stream and the hydrogen rich gas. Alternatively, either fluid can be of sufficient pressure so as to not require such devices. The reforming zone 16 can further include other equipment or vessels, such as other heaters, a recycle gas compressor, other separator vessels, and additional reactors. Alternatively, the reforming reactors 36 can be placed in single operation. Regardless of the design of reforming apparatus 34, hydrocarbon molecules entering the reforming apparatus 34 in the liquid hydrocarbon feedstock stream 24 are rearranged or restructured into more complex molecular shapes having higher octane values during reforming. The reformer effluent stream 26 containing the higher octane components and other unconverted components exits the reforming zone 16 in line 52.

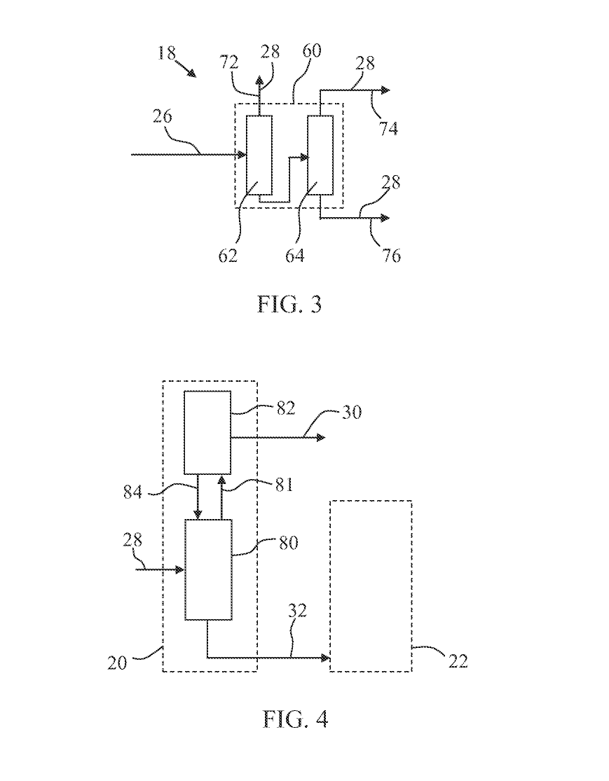

FIG. 3 illustrates further processing performed in the separation zone 18. As shown, the separation zone 18 includes a separation apparatus 60. The exemplary separation apparatus 60 may include one or more distillation columns, such as distillation columns 62 and 64. Although two distillation columns 62 and 64 are depicted, one or more than two distillation columns may be operated in series and/or in a parallel. The distillation columns 62 and 64 separate the components of the reformer effluent stream 26 received from the reforming zone 16 to produce one or more separated reformate streams 28 in lines 72, 74 and 76. Depending on the hydrocarbon feedstock composition and the operation of the separation zone 18, one of the reformate streams 28 will include normal hexane which can be processed further to create the normal hexane product stream 12 (of FIG. 1). In an embodiment in which components lighter than normal hexane are removed in line 72 and components heavier than normal hexane are removed in line 76, the normal hexane will be removed in the reformate stream 28 through line 74.

In FIG. 4, the reformate stream 28 is processed and aromatics are removed therefrom in accordance with an embodiment herein. As shown, the aromatic extraction zone 20 of the apparatus 10 receives the reformate stream 28 from the separation zone 18. Specifically, the reformate stream 28 is delivered to an aromatic extraction unit 80. The aromatic extraction unit 80 extracts aromatics such as benzene, toluene, and xylene to form an extract stream 81. An exemplary aromatic extraction unit 80 uses an extractive distillation flow scheme with a highly selective solvent. For example, sulfolane solvents are capable of providing a sharp separation between aromatics and non-aromatics and may be used. Solvent typically exits the aromatic extraction unit 80 with the extract stream 81. The extract stream 81 is fed to a separator 82 such as a distillation column where the solvent is separated from the aromatics through distillation and returned to the aromatic extraction unit 80 as recycled solvent 84. The aromatic stream 30 exits the separator 82 and aromatic extraction zone 20.

As shown, by removing aromatics from the reformate stream 28, the aromatic extraction unit 80 forms a raffinate stream 32. The raffinate stream 32 typically includes normal paraffins, such as hexane, and cycloparaffins, such as cyclohexane and methylcyclopentane. The raffinate stream 32 exits the aromatic extraction zone 20 and is delivered to the normal hexane isolation zone 22 where the normal hexane product is isolated.

FIG. 5 illustrates an embodiment of the normal hexane isolation zone 22. In FIG. 5, the normal hexane isolation zone 22 includes one or more fractionation columns 90 and 92. As illustrated, fractionation column 90 fractionates the raffinate stream 32 and removes an overhead or light stream 94, including components lighter than normal hexane, such as C5s and lighter C6 isomers including methylpentanes and dimethylbutanes. Then, fractionation column 92 fractionates a remaining raffinate stream 96 to remove a bottom or heavy stream 98, including components heavier than normal hexane, such as C6 naphthenes and C7s. As a result of the fractionation process, the normal hexane product stream 12 exits the normal hexane isolation zone 22 with a desired normal hexane concentration.

Although only two fractionation columns 90 and 92 are depicted in FIG. 5, it should be understood that the normal hexane isolation zone 22 can further include other equipment or vessels, such as one or more heaters, compressors, pumps and additional columns or separation units. Also, the normal hexane isolation zone 22 may comprise a divided wall column for performing the separation. Further, while FIG. 5 illustrates the light stream being separated first, it is contemplated that the light stream 94 be separated after the heavy stream 98 is separated. In either case, the normal hexane product stream 12 is formed with the desired concentration of normal hexane. For example, the normal hexane product stream 12 may include at least about 40 wt % normal hexane, for example at least about 50 wt % normal hexane, such as about 55 wt % normal hexane. The light stream 94 and/or heavy stream 98 may be introduced to the reforming zone 16 for further processing.

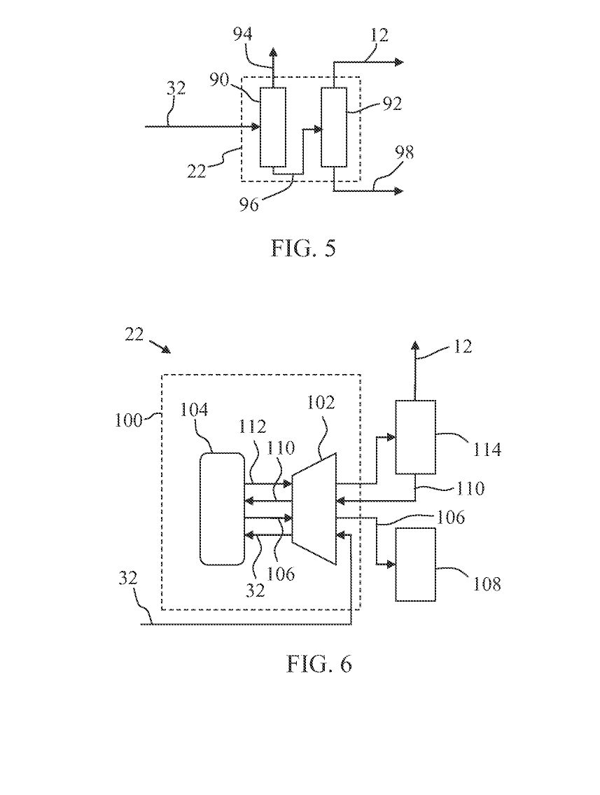

FIG. 6 illustrates another embodiment of the normal hexane isolation zone 22. In FIG. 6, the raffinate stream 32 is delivered to a normal hexane isolation zone 22 including a separator 100 with a rotary valve 102 for separation in a simulated moving bed process. As shown, the separator 100 includes an adsorbent chamber 104 loaded with a molecular sieve or other suitable adsorbent and in communication with the rotary valve 102. The raffinate stream 32 is delivered to the adsorbent chamber 104 through the rotary valve 102. Normal paraffins are adsorbed by the molecular sieve while isoparaffins are not adsorbed. By selectively opening and closing the rotary valve 102, the isoparaffins exit the adsorbent chamber 104 in a raffinate stream 106 which is delivered to a raffinate column 108 where they are processed further, and from which they can be delivered to the reforming zone 16. The rotary valve 102 provides for selectively flowing desorbent 110 into the adsorbent chamber 104 to remove the adsorbed normal paraffins therefrom in an extract stream 112. The extract stream 112 is delivered to an extract column 114 where normal hexane is isolated in the normal hexane product stream 12 and desorbent 110 is separated and returned to the adsorbent chamber 104 through the rotary valve 102.

The adsorbent processing of the normal hexane isolation zone 22 in FIG. 6 provides for a normal hexane product stream 12 having the desired normal hexane concentration—such as at least about 40 wt % normal hexane, for example at least about 50 wt % normal hexane or about 55 wt % normal hexane. Higher concentrations can be achieved with a decreasing yield of normal hexane. Although only the rotary valve 102, adsorbent chamber 104, and columns 108 and 114 are depicted, it should be understood that the normal hexane isolation zone 22 can further include other equipment or vessels, such as one or more heaters, compressors, pumps and additional valves, chambers or separation units.

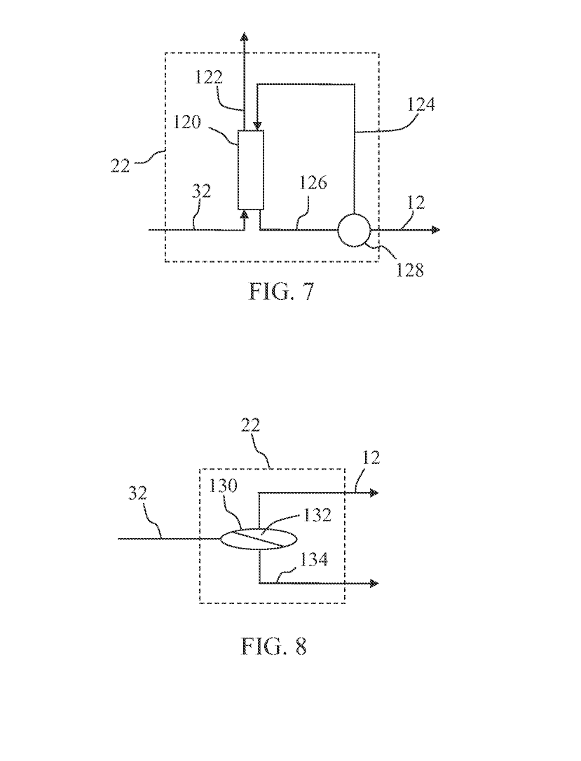

FIG. 7 illustrates an embodiment of the normal hexane isolation zone 22 which utilizes a fixed bed adsorption system. As shown, the raffinate stream 32 is delivered to an adsorbent chamber 120 which holds a molecular sieve or other suitable adsorbent. A typical molecular sieve is a crystalline zeolite having uniform pore dimensions of the same order of magnitude as the size of individual hydrocarbon molecules. The molecule sieve has pore openings in the crystalline structure that are sized to allow molecules of normal paraffins to pass through the pore openings and into the internal crystal cavity where they are retained. Non-normal hydrocarbons, having larger molecular diameters are excluded from entering the crystal cavities through the pore openings.

As a result, normal paraffins are adsorbed by the adsorbent in the adsorbent chamber 120 while non-normal hydrocarbons remain non-adsorbed and exit the adsorbent chamber 120 in a raffinate stream 122. The raffinate stream 122 may undergo further processing such as in a stripper to isolate desirable components or to recycle components, such as to the reforming zone 16. In order to remove the adsorbed normal paraffins from the adsorbent chamber 120, flow of the raffinate stream 32 is stopped and a desorbent stream 124 is selectively fed in a counterflow direction into the adsorbent chamber 120. The desorbent stream 124 is formed by a non-adsorbable medium, such as hydrogen, for removing the adsorbed normal paraffins from the adsorbent. The desorbed normal paraffins and the desorbent exit the adsorbent chamber 120 in a desorbed stream 126. Then the desorbed stream 126 passes through a separator 128 where the desorbent is removed and recycled in the desorbent stream 124. After separation, the normal hexane product stream 12 exits the normal hexane isolation zone 22.

As a result of the fixed bed adsorption process of FIG. 7, the normal paraffins are isolated into the normal hexane product stream 12 at the desired concentration. Although only the adsorbent chamber 120 and separator 128 are depicted, it should be understood that the normal hexane isolation zone 22 can further include other equipment or vessels, such as one or more heaters, a recycle compressor, pumps and additional chambers or separators.

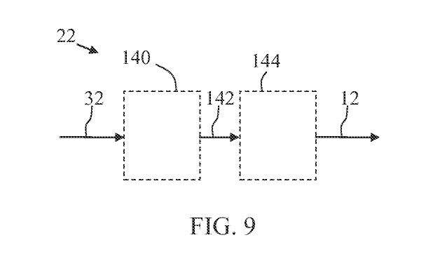

In FIG. 8, the normal hexane isolation zone 22 provides for separation of normal hexane through a selectively permeable membrane. As shown, the raffinate stream 32 is delivered to the retentate side of membrane 130. The membrane 130 is selective for the permeation of normal paraffins. Thus, the permeate 132 passing through the membrane 130 is concentrated in normal hexane and forms the normal hexane product stream 12. Non-normal hydrocarbons form the retentate 134 which can be recycled to the reforming zone 16 of FIG. 1.

FIG. 9 illustrates the use of a normal hexane isolation zone 22 that utilizes a plurality of the separation processes described in FIGS. 5-8. Specifically, the normal hexane isolation zone 22 of FIG. 9 provides an upstream isolation zone 140 that receives the raffinate stream 32 and isolates normal hexane in an effluent 142. The effluent 142 is delivered to a downstream isolation zone 144 where further isolation of normal hexane occurs. The normal hexane product stream 12 exits the downstream isolation zone 144 with the desired normal hexane concentration. It is contemplated that the upstream isolation zone 140 includes the apparatus of and performs the process of one of the normal hexane isolation zones 22 described in FIGS. 5-8. Likewise, the downstream isolation zone 144 also includes the apparatus of and performs the process of one of the normal hexane isolation zones 22 described in FIGS. 5-8.

As an example, the upstream isolation zone 140 may perform a simulated moving bed separation process as described in FIG. 6 and deliver its effluent 142 to the downstream isolation zone 144 comprised of a column or multiple columns in accordance with FIG. 5. It is contemplated that the isolation zones 140 and 144 can be arranged to provide the most cost effective processing while forming a sufficient yield of normal hexane product stream 12 having the desired concentration of normal hexane. While two isolation zones 140 and 144 are illustrated in FIG. 9, it is further contemplated that three or more isolation zones be used to achieve the desired normal hexane product concentration and yield.

Accordingly, methods and apparatuses for recovering normal hexane from reformate streams have been described. The embodiments described herein provide a reformate product, such as a high octane hydrocarbon stream, and a normal hexane product stream formed from a reformate. While substantially any concentration of normal hexane can be achieved in the normal hexane product, to maximize yield and value of the normal hexane product it typically has a concentration of more than about 40 wt % normal hexane, for example more than about 50 wt % normal hexane, such as about 55 wt % normal hexane.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. Various changes may be made in the function and arrangement of elements described in an exemplary embodiment without departing from the scope of the disclosure as set forth in the appended claims.

1. A method for recovering normal hexane from a reformate stream comprising:
extracting aromatics from the reformate stream to form an aromatic extract stream and a raffinate stream; and
separating normal hexane from the raffinate stream to form a normal hexane product stream wherein separating the normal hexane from the raffinate stream comprises passing the raffinate stream through a molecular sieve and adsorbing normal hexane in the molecular sieve to form a non-normal paraffin stream and wherein separating normal hexane from the raffinate stream forms the normal hexane product stream with a composition of at least 45 wt % normal hexane.

2. The method of claim 1 wherein separating the normal hexane from the raffinate stream comprises fractionating the raffinate stream to isolate the normal hexane.

3. The method of claim 2 wherein fractionating the raffinate stream comprises forming the normal hexane product stream, a light stream comprising pentanes and light hexane isomers, and a heavy stream comprising naphthenes and heptanes.

4. The method of claim 3 further comprising:
creating the reformate stream in an upstream reforming apparatus; and
introducing the heavy stream to the upstream reforming apparatus.

5. (canceled)

6. The method of claim 1 further comprising:
creating the reformate stream in an upstream reforming apparatus; and
introducing the non-normal paraffin stream to the upstream reforming apparatus.

7. The method of claim 1 wherein other normal paraffins are adsorbed in the molecular sieve and wherein separating the normal hexane from the raffinate stream further comprises:
releasing a stream of normal paraffins from the molecular sieve; and
fractionating the stream of normal paraffins to isolate the normal hexane.

8. The method of claim 1 wherein separating the normal hexane from the raffinate stream comprises passing the raffinate stream through a membrane to separate normal hexane from a non-normal paraffin stream.

9. (canceled)

10. A method for forming a heavy aromatic rich product and a normal hexane product comprising:
reforming a hydrocarbon stream to form the heavy aromatic rich product and a light reformate stream;
separating non-aromatics from the light reformate stream wherein separating non-aromatics from the light reformate stream comprises extracting aromatics from the light reformate stream to form an aromatic extract stream and a raffinate stream, wherein the raffinate stream includes the non-aromatics and wherein isolating the normal hexane comprises passing the non-aromatics through a molecular sieve and adsorbing normal hexane in the molecular sieve to form a non-normal paraffin stream; and
isolating normal hexane from the non-aromatics to form a normal hexane product stream and wherein isolating normal hexane from the non-aromatics forms the normal hexane product stream with a composition of at least 45 wt % normal hexane.

11. (canceled)

12. The method of claim 10 wherein isolating the normal hexane comprises fractionating the non-aromatics to isolate the normal hexane.

13. The method of claim 12 wherein fractionating the non-aromatics comprises forming the normal hexane product stream, a light stream comprising pentanes and light hexane isomers, and a heavy stream comprising naphthenes and heptanes.

14. The method of claim 13 wherein reforming comprises reforming the hydrocarbon stream in an upstream reforming apparatus and wherein the method further comprises introducing the heavy stream to the upstream reforming apparatus.

15. (canceled)

16. The method of claim 10 wherein other normal paraffins are adsorbed in the molecular sieve and isolating the normal hexane further comprises:
releasing a stream of normal paraffins from the molecular sieve; and
fractionating the stream of normal paraffins to isolate the normal hexane.

17. The method of claim 10 wherein isolating the normal hexane comprises passing the non-aromatics through a membrane to separate normal hexane from a non-normal paraffin stream.

18. (canceled)

19. An apparatus for recovering normal hexane from a reformate stream comprising:
a reforming unit configured to form the reformate stream;
an aromatic extraction unit configured to extract aromatics from the reformate stream to form an aromatic extract stream and a raffinate stream; and
a separation unit configured to isolate normal hexane in a normal hexane product stream.

20. The apparatus of claim 19 wherein the separation unit comprises a fractionation column, a molecular sieve and/or a membrane.
